(19) United States
(12) Patent Application Publication
(10) Pub. No.: US 2019/0175646 A1
(43) Pub. Date: Jun. 13, 2019

(54) METHODS AND COMPOSITIONS RELATING TO THE TREATMENT OF BENIGN SKIN TUMORS

(71) Applicant: THE GENERAL HOSPITAL CORPORATION D/B/A MASSACHUSETTS GENERAL HOSPITAL, Boston, MA (US)

(72) Inventors: Anna MANDINOVA, Newton, MA (US); Victor Allen NEEL, Providence, RI (US)

(73) Assignee: THE GENERAL HOSPITAL CORPORATION D/B/A MASSACHUSETTS GENERAL HOSPITAL, Boston, MA (US)

(21) Appl. No.: 15/779,778

(22) PCT Filed: Nov. 28, 2016
(86) PCT No.: PCT/US16/63847

§ 371 Date: May 29, 2018

Related U.S. Application Data
(60) Provisional application No. 62/261,410, filed on Dec. 1, 2015.
(60) Provisional application No. 62/303,412, filed on Mar. 4, 2016.

(51) Int. Cl. A61K 33/30; A61K 33/34; A61K 31/145; A61K 31/44; A61K 9/00; A61P 35/00

(52) U.S. Cl. CPC A61K 33/30; A61K 33/34; A61P 35/00; A61K 31/44; A61K 9/0014; A61K 31/145

(57) ABSTRACT

Described herein are methods and compositions relating to the treatment of benign skin tumors and/or malformations (e.g., seborrheic keratosis) by administering a proteasomal inhibitor and/or cationic inhibitor in combination with Zn2+ or Cu2+.

### CROSS-REFERENCE TO RELATED APPLICATIONS

This application claims benefit under 35 U.S.C. § 119(e) of U.S. Provisional Application Nos. 62/261,410 filed Dec. 1, 2015 and 62/303,412 filed Mar. 4, 2016, the contents of which are incorporated herein by reference in their entirety.

### TECHNICAL FIELD

The technology described herein relates to the treatment of benign skin tumors and/or malformations, e.g. by administering a metal ion and proteasomal inhibitor.

### BACKGROUND

Seborrheic keratoses (SKs) are common, benign age-related skin tumors. They can be mimic more serious skin conditions such as squamous cell carcinoma and melanoma. Many patients develop multiple lesions and they are a serious cosmetic concern. Existing clinical interventions are limited to surgical removal of the affected skin area.

### SUMMARY

As described herein, the inventors have found that cationic ionophores or proteasomal inhibitors administered with metal ions (e.g. Cu2+ or Zn2+), demonstrate a surprising ability to reduce the viability of SK cells, while being nontoxic to healthy cells. Accordingly, described herein are methods and compositions relating to the treatment of benign skin tumors and/or malformations such as seborrheic keratosis by administering 1) proteasomal inhibitors and metal ions or 2) cationic ionophores with or without metal ions.

In one aspect, described herein is a method of treating a benign skin tumor and/or malformation in a subject in need thereof, the method comprising administering a proteasomal inhibitor and Zn2+ or Cu2+ to the subject. In some embodiments of any of the aspects, the proteasomal inhibitor is a cationic ionophore.

In one aspect, described herein is a method of treating a benign skin tumor and/or malformation in a subject in need thereof, the method comprising administering a cationic ionophore to the subject.

In some embodiments of any of the aspects, the cationic ionophore is disulfiram (DSF) or pyrithione. In some embodiments of any of the aspects, the cationic ionophore is not complexed with Zn2+ or Cu2+. In some embodiments of any of the aspects, the cationic ionophore administered with Zn2+ or Cu2+. In some embodiments of any of the aspects, the proteasomal inhibitor and/or cationic ionophore is complexed with Zn2+ or Cu2+. In some embodiments of any of the aspects, the cationic ionophore is complexed with Zn2+ or Cu2+ at a ratio of about 1:10 or greater. In some embodiments of any of the aspects, the cationic ionophore is complexed with Zn2+ or Cu2+ at a ratio of about 1:1 or greater. In some embodiments of any of the aspects, the cationic ionophore is complexed with Zn2+ or Cu2+ at a ratio of about 1:1 to about 10:1.

In some embodiments of any of the aspects, the cationic ionophore is DSF complexed with or administered with Cu2+.

In some embodiments of any of the aspects, the benign skin tumor and/or malformation is seborrheic keratosis; acquired acanthosis nigricans; Becker's nevus; or congenital non-epidermolytic epidermal nevi.

In some embodiments of any of the aspects, the proteasomal inhibitor and/or cationic ionophore is administered topically. In some embodiments of any of the aspects, the proteasomal inhibitor and/or cationic ionophore is administered intradermally. In some embodiments of any of the aspects, the proteasomal inhibitor and/or cationic ionophore is administered iontophoretically. In some embodiments of any of the aspects, the proteasomal inhibitor and/or cationic ionophore is administered systemically.

In some embodiments of any of the aspects, the proteasomal inhibitor and/or cationic ionophore is administered at a dose that does not substantially affect the survival of normal keratinocytes or normal skin explants.

### BRIEF DESCRIPTION OF THE DRAWINGS

FIG. 1 depicts a graph in which SK cells were treated with DSF and CuCl₂ (indicated concentrations) for 48 hours and relative cellular viability was measured using Alamar blue (absorbance based) assay. A443654 Akt inhibitor was used as a control.

FIG. 2 depicts a graph in which SK cells and normal human keratinocytes were treated with DSF and CuCl₂ (indicated concentrations) for 48 hours and relative cellular viability was measured using Alamar blue (absorbance based) assay. A443654 Akt inhibitor was used as a control.

FIG. 3 depicts a graph of melanocyte viability. DSF10 uM+Cu10 uM is toxic to SK cells, but safe on keratinocytes and melanocytes. DSF10 uM+Zn10 uM is toxic to SK cell and melanocytes, but safe on keratinocytes.

FIG. 4 depicts a graph of SK cell viability after treatment with DSF and/or Cu²⁺. A443654 Akt inhibitor was used as a control.

FIG. 5 depicts a graph of SK cell viability after treatment with DSF and/or Zn²⁺. A443654 Akt inhibitor was used as a control.

FIG. 6 depicts a graph of normal keratinocyte viability after treatment with DSF and/or Cu²⁺. A443654 Akt inhibitor was used as a control.

FIG. 7 depicts a graph of normal keratinocyte viability after treatment with DSF and/or Zn²⁺. A443654 Akt inhibitor was used as a control.

FIGS. 8 and 9 depict graphs of SK cell viability after treatment with pyrithione and/or Cu²⁺. A443654 Akt inhibitor was used as a control. Cells from different donors were used for each experiment

FIG. 10 depicts a graph of normal keratinocyte viability after treatment with pyrithione and/or Cu²⁺. A443654 Akt inhibitor was used as a control.

FIG. 11 depicts a graph of viability of normal human skin (N) or seborrheic keratosis (SK) explants after treatment with DSF and/or Cu²⁺.

### DETAILED DESCRIPTION

Treatments for a number of benign skin tumors and/or malformations are currently extremely limited. For example, the only treatment currently available in the clinic for seborrheic keratosis is mechanical removal of the tumors, such as laser or surgery. These treatments relate to the administration of metal ions (e.g., Cu²⁺ and/or Zn²⁺) in combination with proteasomal inhibitors, e.g., cationic ionophores. The transport of the metal ion into the cell with the proteasomal inhibitor (e.g. cationic ionophore) provides a surprising increase of efficacy and specificity as compared to the inhibitor in the absence of the metal ion.

In one aspect of any of the embodiments, described herein is a method of treating a benign skin tumor and/or malformation in a subject in need thereof, the method comprising administering a proteasomal inhibitor and Zn²⁺ and/or Cu²⁺ to the subject.

As used herein, a “benign skin tumor” and/or “benign skin malformation” refers to any non-cancerous tumor, growth, and/or malformation of the skin. Non-limiting examples of benign skin tumors and/or malformations can include, e.g., seborrheic keratosis; acquired acanthosis nigricans; Becker's nevus; and congenital non-epidermolytic epidermal nevi. In some embodiments of any of the aspects described herein, the method relates to treatment of seborrheic keratosis; acquired acanthosis nigricans; Becker's nevus; and/or congenital non-epidermolytic epidermal nevi.

As used herein, the term “inhibitor” refers to an agent which can decrease the expression and/or activity of the targeted expression product (e.g. mRNA encoding the target or a target polypeptide), e.g. by at least 10% or more, e.g. by 10% or more, 50% or more, 70% or more, 80% or more, 90% or more, 95% or more, or 98% or more. The efficacy of an inhibitor of, for example, the proteasome, e.g. its ability to decrease the level and/or activity of one or more proteasome proteins can be determined, e.g. by measuring the level of a protein degradation by the proteasome and/or by measuring the level of a proteasome protein (or its mRNA). Methods for measuring the level of a given mRNA and/or polypeptide are known to one of skill in the art, e.g. RT-PCR with primers can be used to determine the level of RNA and Western blotting with an antibody can be used to determine the level of a polypeptide. The activity of, e.g. the proteasome can be determined using methods known in the art, e.g. using a commercial kit (e.g., Cat No. 23623; Thermo Scientific). In some embodiments, an inhibitor can be an inhibitory nucleic acid; an aptamer; an antibody reagent; an antibody; or a small molecule.

As used herein, “proteasomal inhibitor” refers to a compound that inhibits one or more activities of a proteasome. In some embodiments of any of the aspects, the proteasomal inhibitor is a cationic ionophore. Non-limiting examples of proteasomal inhibitors can include, e.g., lactacystin, bortezomib, epigallocatechin-3-gallate, salinosporamide A, carfilzomib, ONX 0912, CEP-18770, MLN9708, epoxomicin, MG132, Ixazomib. Additional non-limiting example of proteasomal inhibitors can be found, e.g., in U.S. Pat. No. 8,809,283; US Patent Publication 2011/0009332; International Patent Publications WO 2014/182744; WO1999/037666; and European Patent 1895971; each of which is incorporated by reference herein in its entirety.

In one aspect of any of the embodiments, described herein is a method of treating a benign skin tumor and/or malformation in a subject in need thereof, the method comprising administering a cationic ionophore to the subject. As used herein, “cationic ionophore” refers to a compound that can transport a cation across a lipid bilayer (e.g., a cell membrane). In some embodiments of any of the aspects, the cationic ionophore can transport Cu²⁺ and/or Zn²⁺.

Non-limiting examples of cationic ionophores include, e.g., disulfiram (DSF) and pyrithione. In some embodiments of any of the aspects, the method relates to administration of disulfiram (DSF) and/or pyrithione. In some embodiments of any of the aspects, the cationic ionophore is not clioquinol. In some embodiments of any of the aspects, the cationic ionophore is a Dithiocarbamate (e.g., pyrrolidine dithiocarbamate, diethyldithiocarbamate, dimethyldithiocarbamate, carbamodithioic acid, prolinedithiocarbamate, pyrrolidine dithiocarbamate, dimethyldithiocarbamate, diethyldithiocarbamate ion, and dibenzyldithiocarbamate). In some embodiments of any of the aspects, the cationic ionophore is a heterocyclic amine (e.g., 5,7-Diiodo-8-hydroxyquinoline, and 8-Hydroxyquinoline); or a vitamin including, but not limited to, Vitamin E and Vitamin A.

In some embodiments of any of the aspects, the cationic ionophore is not complexed with a metal ion, e.g., Zn²⁺ and/or Cu²⁺ at administration. In some embodiments of any of the aspects, the cationic ionophore is not administered with a metal ion, e.g., Zn²⁺ and/or Cu²⁺. In some embodiments of any of the aspects, the cationic ionophore is complexed with a metal ion, e.g., Zn²⁺ and/or Cu²⁺ at administration. In some embodiments of any of the aspects, the cationic ionophore is administered with a metal ion, e.g., Zn²⁺ and/or Cu²⁺.

In some embodiments of any of the aspects, the proteasomal inhibitor and/or cationic ionophore is complexed with Zn²⁺ or Cu²⁺ at a ratio of about 1:10 or greater at administration. In some embodiments of any of the aspects, the proteasomal inhibitor and/or cationic ionophore is complexed with Zn²⁺ or Cu²⁺ at a ratio of 1:10 or greater at administration. In some embodiments of any of the aspects, the proteasomal inhibitor and/or cationic ionophore is complexed with Zn²⁺ or Cu²⁺ at a ratio of about 1:1 or greater at administration. In some embodiments of any of the aspects, the proteasomal inhibitor and/or cationic ionophore is complexed with Zn²⁺ or Cu²⁺ at a ratio of 1:1 or greater at administration. In some embodiments of any of the aspects, the proteasomal inhibitor and/or cationic ionophore is complexed with Zn²⁺ or Cu²⁺ at a ratio of from about 1:1 to about 10:1 at administration. In some embodiments of any of the aspects, the proteasomal inhibitor and/or cationic ionophore is complexed with Zn²⁺ or Cu²⁺ at a ratio of from 1:1 to 10:1 at administration. In some embodiments of any of the aspects, the proteasomal inhibitor and/or cationic ionophore is complexed with Zn²⁺ or Cu²⁺ at a ratio of from about 1:10 to about 10:1 at administration. In some embodiments of any of the aspects, the proteasomal inhibitor and/or cationic ionophore is complexed with Zn²⁺ or Cu²⁺ at a ratio of from 1:10 to 10:1 at administration.

In some embodiments of any of the aspects, the proteasomal inhibitor and/or cationic ionophore is administered with Zn²⁺ or Cu²⁺ at a ratio of about 1:10 or greater. In some embodiments of any of the aspects, the proteasomal inhibitor and/or cationic ionophore is administered with Zn²⁺ or Cu²⁺ at a ratio of 1:10 or greater. In some embodiments of any of the aspects, the proteasomal inhibitor and/or cationic ionophore is administered with Zn²⁺ or Cu²⁺ at a ratio of about 1:1 or greater. In some embodiments of any of the aspects, the proteasomal inhibitor and/or cationic ionophore is administered with Zn²⁺ or Cu²⁺ at a ratio of 1:1 or greater. In some embodiments of any of the aspects, the proteasomal inhibitor and/or cationic ionophore is administered with Zn²⁺ or Cu²⁺ at a ratio of from about 1:1 to about 10:1. In some embodiments of any of the aspects, the proteasomal inhibitor and/or cationic ionophore is administered with Zn²⁺ or Cu²⁺ at a ratio of from 1:1 to 10:1. In some embodiments of any of the aspects, the proteasomal inhibitor and/or cationic ionophore is administered with Zn²⁺ or Cu²⁺ at a ratio of from about 1:10 to about 10:1. In some embodiments of any of the aspects, the proteasomal inhibitor and/or cationic ionophore is administered with Zn²⁺ or Cu²⁺ at a ratio of from 1:10 to 10:1.

In some embodiments, the cationic ionophore is disulfram (DSF) and the metal ion is Cu²⁺.

In some embodiments of any of the aspects, the proteasomal inhibitor and/or cationic ionophore is complexed with Zn²⁺ at administration. In some embodiments of any of the aspects, the proteasomal inhibitor and/or cationic ionophore is complexed with Zn²⁺ and not with Cu²⁺ at administration. In some embodiments of any of the aspects, the proteasomal inhibitor and/or cationic ionophore is adminstered with Zn²⁺. In some embodiments of any of the aspects, the proteasomal inhibitor and/or cationic ionophore is administered with Zn²⁺ and not with Cu²⁺.

In some embodiments of any of the aspects, the proteasomal inhibitor and/or cationic ionophore is complexed with Cu²⁺ at administration. In some embodiments of any of the aspects, the proteasomal inhibitor and/or cationic ionophore is complexed with Cu²⁺ and not with Zn²⁺ at administration. In some embodiments of any of the aspects, the proteasomal inhibitor and/or cationic ionophore is adminstered with Cu²⁺. In some embodiments of any of the aspects, the proteasomal inhibitor and/or cationic ionophore is administered with Cu²⁺ and not with Zn²⁺.

In some embodiments of any of the aspects, the proteasomal inhibitor and/or cationic ionophore is complexed with Zn²⁺ and Cu²⁺ at administration. In some embodiments of any of the aspects, the proteasomal inhibitor and/or cationic ionophore is administered with Zn²⁺ and Cu²⁺.

In some embodiments of any of the aspects, the metal ion and cationic ionophore are administered separately and the metal ion is administered and/or applied after the cationic ionophore is administered and/or applied. In some embodiments of any of the aspects, the metal ion and cationic ionophore are administered separately and the cationic ionophore is administered and/or applied after the metal ion is administered and/or applied. In some embodiments of any of the aspects, the metal ion and cationic ionophore are stored separately and mixed immediately prior to or at the time of administration/application. In some embodiments of any of the aspects, provided herein is a kit with a first container comprising the metal ion and a second container comprising the cationic ionophore. In some embodiments of any of the aspects, the kit further comprises instructions to mix the two compositions and immediately administer/apply the mixture. In some embodiments of any of the aspects, the kit further comprises instructions to administer/apply the two compositions successively.

In some embodiments of any of the aspects, the proteasomal inhibitor and/or cationic ionophore is administered topically. In some embodiments of any of the aspects, the proteasomal inhibitor and/or cationic ionophore is administered intradermally. In some embodiments of any of the aspects, the proteasomal inhibitor and/or cationic ionophore is administered iontophoretically. In some embodiments of any of the aspects, the proteasomal inhibitor and/or cationic ionophore is administered systemically.

In some embodiments of any of the aspects, the proteasomal inhibitor and/or cationic ionophore is administered at a dose that does not substantially affect the survival of normal keratinocytes or normal skin explants.

In some embodiments of any of the aspects, the methods described herein relate to treating a subject having or diagnosed as having, e.g., seborrheic keratosis with a metal ion and proteasomal inhibitor. Subjects having seborrheic keratosis can be identified by a physician using current methods of diagnosing seborrheic keratosis. Symptoms and/or complications of seborrheic keratosis which characterize these conditions and aid in diagnosis are well known in the art and include but are not limited to, presence of one or more waxy and/or wart-like growths ranging in color from light tan to black. Tests that may aid in a diagnosis of, e.g. seborrheic keratosis include, but are not limited to, skin biopsies. A family history of seborrheic keratosis, or exposure to risk factors for seborrheic keratosis (e.g. being greater than 40 years of age) can also aid in determining if a subject is likely to have seborrheic keratosis or in making a diagnosis of seborrheic keratosis.

Seborrheic keratoses (SKs) are the most common benign epithelial tumors in humans. The etiology of SKs are unknown but they exhibit histologic evidence of increased proliferation of keratinocytes. These lesions have an increased rate of apoptosis and several studies show that their incidence increases with age. Some studies have found that 88% of individuals over age 64 have at least one SK. SKs are characterized as a dull hyperkeratotic macule that evolves to a papulonodular lesion. They can appear as pale brown, pink, tan or brown in color and the surface can become warty or verrucous. The size varies from 5 mm to several centimeters and a classic “stuck on” appearance is observed. SKs never progress to malignant tumors.

Seborrheic keratoses can be easily diagnosed visually by one of skill in the art of medicine, particularly dermatology. A seborrheic keratosis is typically a brown, black or pale growth found on the back, shoulders, face or chest. In general, a seborrheic keratosis is slightly elevated above the skin surface and can appear waxy or scaly. In some cases, a seborrheic keratosis can resemble a wart, an actinic keratosis, or skin cancer. If a doctor suspects skin cancer, a biopsy can be performed to confirm that a growth is a seborrheic keratosis. Such methods are routine to those of skill in the art.

The compositions and methods described herein can be administered to a subject having or diagnosed as having, e.g., seborrheic keratosis. In some embodiments of any of the aspects, the methods described herein comprise administering an effective amount of compositions described herein, e.g. a proteasomal inhibitor and metal ion to a subject in order to alleviate a symptom of a benign skin tumor and/or malformation. As used herein, “alleviating a symptom” is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of an active ingredient needed to alleviate at least one or more symptom of the disease or disorder, and relates to a sufficient amount of pharmacological composition to provide the desired effect. The term “therapeutically effective amount” therefore refers to an amount of the active ingredient that is sufficient to provide a particular therapeutic effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for tumor size and/or growth, among others. The dosage can be determined by a physician and adjusted, as necessary, to suit observed effects of the treatment.

In one aspect of any of the embodiments, described herein is a composition, e.g., a pharmaceutical composition comprising a proteasomal inhibitor and a metal ion. In some embodiments of any of the aspects, the ratio of the proteasomal inhibitor:metal ion is about 1:10 or greater. In some embodiments of any of the aspects, the ratio of the proteasomal inhibitor:metal ion is 1:10 or greater. In some embodiments of any of the aspects, the ratio of the proteasomal inhibitor:metal ion is about 1:1 or greater. In some embodiments of any of the aspects, the ratio of the proteasomal inhibitor:metal ion is 1:1 or greater. In some embodiments of any of the aspects, the ratio of the proteasomal inhibitor:metal ion is from about 1:1 to about 10:1. In some embodiments of any of the aspects, the ratio of the proteasomal inhibitor:metal ion is from 1:1 to 10:1. In some embodiments of any of the aspects, the ratio of the proteasomal inhibitor:metal ion is from about 1:10 to about 10:1. In some embodiments of any of the aspects, the ratio of the proteasomal inhibitor:metal ion is from 1:10 to 10:1. In some embodiments, the metal ion is Zn²⁺. In some embodiments, the metal ion is Cu²⁺. In some embodiments, the metal ion is a combination of Cu²⁺ and Zn²⁺. In some embodiments, the metal ion is Zn²⁺ and the composition does not comprise Cu²⁺. In some embodiments, the metal ion is Cu²⁺ and the composition does not comprise Zn²⁺.

In one aspect of any of the embodiments, described herein is a composition, e.g., a pharmaceutical composition comprising a cationic ionophore and a metal ion. In some embodiments of any of the aspects, the ratio of the cationic ionophore:metal ion is about 1:10 or greater. In some embodiments of any of the aspects, the ratio of the cationic ionophore:metal ion is 1:10 or greater. In some embodiments of any of the aspects, the ratio of the cationic ionophore:metal ion is about 1:1 or greater. In some embodiments of any of the aspects, the ratio of the cationic ionophore:metal ion is 1:1 or greater. In some embodiments of any of the aspects, the ratio of the cationic ionophore:metal ion is from about 1:1 to about 10:1. In some embodiments of any of the aspects, the ratio of the cationic ionophore:metal ion is from 1:1 to 10:1. In some embodiments of any of the aspects, the ratio of the cationic ionophore:metal ion is from about 1:10 to about 10:1. In some embodiments of any of the aspects, the ratio of the cationic ionophore:metal ion is from 1:10 to 10:1. In some embodiments, the metal ion is Zn²⁺. In some embodiments, the metal ion is Cu²⁺. In some embodiments, the metal ion is a combination of Cu²⁺ and Zn²⁺. In some embodiments, the metal ion is Zn²⁺ and the composition does not comprise Cu²⁺. In some embodiments, the metal ion is Cu²⁺ and the composition does not comprise Zn²⁺. In some embodiments, the cationic ionophore is disulfram (DSF). In some embodiments, the cationic ionophore is pyrithione. In some embodiments, the cationic ionophore is a combination of pyrithione and disulfram (DSF). In some embodiments, the cationic ionophore is disulfram (DSF) and the metal ion is Cu²⁺.

In one aspect of any of the embodiments, described herein is a composition, e.g., a pharmaceutical composition comprising a cationic ionophore. In some embodiments of any of the aspects, the composition does not comprise a metal ion. In some embodiments of any of the aspects, the composition does not comprise Cu²⁺ or Zn²⁺. In some embodiments, the cationic ionophore is disulfram (DSF). In some embodiments, the cationic ionophore is pyrithione. In some embodiments, the cationic ionophore is a combination of pyrithione and disulfram (DSF).

In some embodiments of any of the aspects, the technology described herein relates to a pharmaceutical composition comprising a proteasomal inhibitor and optionally, a metal ion as described herein, and optionally a pharmaceutically acceptable carrier. In some embodiments of any of the aspects, the active ingredients of the pharmaceutical composition comprise a proteasomal inhibitor, cationic ionophore, and/or metal ion as described herein. In some embodiments of any of the aspects, the active ingredients of the pharmaceutical composition consist essentially of a proteasomal inhibitor, cationic ionophore, and/or metal ion as described herein. In some embodiments of any of the aspects, the active ingredients of the pharmaceutical composition consist of a proteasomal inhibitor, cationic ionophore, and/or metal ion as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments of any of the aspects, the carrier inhibits the degradation of the active agent, e.g. a proteasomal inhibitor, cationic ionophore, and/or metal ion as described herein.

In some embodiments of any of the aspects, the pharmaceutical composition comprising as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a composition as disclosed herein can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments of any of the aspects, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The methods described herein can further comprise administering a second agent and/or treatment to the subject, e.g. as part of a combinatorial therapy.

In certain embodiments, an effective dose of a composition as described herein can be administered to a patient once. In certain embodiments, an effective dose of a composition can be administered to a patient repeatedly. For systemic administration, subjects can be administered a therapeutic amount of a composition, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments of any of the aspects, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. a tumor by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient. The desired dose or amount of activation can be administered at one time or divided into subdoses, e.g., 2-4 subdoses and administered over a period of time, e.g., at appropriate intervals through the day or other appropriate schedule. In some embodiments of any of the aspects, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition as described herein can be administered over a period of time, such as over a 5 minute, 10 minute, 15 minute, 20 minute, or 25 minute period.

The dosage ranges for the administration of the compositions as described herein, according to the methods described herein depend upon, for example, the form of the active ingredient, its potency, and the extent to which symptoms, markers, or indicators of a condition described herein are desired to be reduced, for example the percentage reduction desired for tumor size. The dosage should not be so large as to cause adverse side effects, such as death of healthy skin cells. Generally, the dosage will vary with the age, condition, and sex of the patient and can be determined by one of skill in the art. The dosage can also be adjusted by the individual physician in the event of any complication.

The efficacy of a composition as described herein in, e.g. the treatment of a condition described herein, can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. tumor size. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. decrease in tumor size). It is well within the ability of one skilled in the art to monitor efficacy of administration and/or treatment by measuring any one of such parameters, or any combination of parameters. Efficacy can be assessed in animal models of a condition described herein, for example treatment of seborrheic keratosis. When using an experimental animal model, efficacy of treatment is evidenced when a statistically significant change in a marker is observed, e.g. tumor size.

In vitro and animal model assays are provided herein which allow the assessment of a given dose of a composition as described herein. By way of non-limiting example, the effects of a dose of a composition described herein can be assessed by assessing the viability of explants of normal skin and/or SK cells.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments of any of the aspects, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments of any of the aspects, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments of any of the aspects, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a benign skin tumor and/or malformation. A subject can be male or female.

A subject can be one who has been previously diagnosed with or identified as suffering from or having a condition in need of treatment (e.g. a benign skin tumor and/or malformation) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

As used herein an “antibody” refers to IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site on an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like. In some embodiments, an antibody reagent can be a single domain antibody. In some embodiments of any of the aspects, the antibody reagent can be a single chain antibody reagent, e.g., one which, as a single polypeptide chain, can specifically bind the target antigen (e.g. nanobodies, VNA, and VHH).

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

Additionally, and as described herein, a recombinant humanized antibody, e.g., single domain antibody (VHH) can be further optimized to decrease potential immunogenicity, while maintaining functional activity, for therapy in humans. In this regard, functional activity means a polypeptide capable of displaying one or more known functional activities associated with a recombinant antibody or antibody reagent thereof as described herein. Such functional activities include, e.g. the ability to bind to a target.

Inhibitors of the expression of a given gene can be an inhibitory nucleic acid. In some embodiments, the inhibitory nucleic acid is an inhibitory RNA (iRNA). As used herein, the term “iRNA” refers to any type of interfering RNA, including but are not limited to RNAi, siRNA, shRNA, endogenous microRNA and artificial microRNA. Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA as that term is defined herein, and which mediates the targeted cleavage of an RNA transcript, e.g., via an RNA-induced silencing complex (RISC) pathway. In one embodiment, an iRNA as described herein effects inhibition of the expression and/or activity of a target gene described herein. In certain embodiments, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA.

In some embodiments, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

In yet another embodiment, the RNA of an iRNA, e.g., a dsRNA, is chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. seborrheic keratosis. The term “treating” includes reducing or alleviating at least one adverse effect or symptom of a condition, disease or disorder associated with a condition. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:

1. A method of treating a benign skin tumor and/or malformation in a subject in need thereof, the method comprising administering a proteasomal inhibitor and Zn2+ or Cu2+ to the subject.
2. The method of paragraph 1, wherein the proteasomal inhibitor is a cationic ionophore.
3. A method of treating a benign skin tumor and/or malformation in a subject in need thereof, the method comprising administering a cationic ionophore to the subject.
4. The method of any of the preceding paragraphs, wherein the cationic ionophore is disulfiram (DSF) or pyrithione.
5. The method of any of the preceding paragraphs, wherein the cationic ionophore is not complexed with Zn2+ or Cu2+.
6. The method of any of the preceding paragraphs, wherein the cationic ionophore administered with Zn2+ or Cu2+.
7. The method of any of the preceding paragraphs, wherein the proteasomal inhibitor and/or cationic ionophore is complexed with Zn2+ or Cu2+.
8. The method of paragraph 6, wherein the cationic ionophore is complexed with Zn2+ or Cu2+ at a ratio of about 1:10 or greater.
9. The method of paragraph 6, wherein the cationic ionophore is complexed with Zn2+ or Cu2+ at a ratio of about 1:1 or greater.
10. The method of paragraph 6, wherein the cationic ionophore is complexed with Zn2+ or Cu2+ at a ratio of about 1:1 to about 10:1.
11. The method of any of the preceding paragraphs, wherein the cationic ionophore is DSF complexed with or administered with Cu2+.
12. The method of any of the preceding paragraphs, wherein the benign skin tumor and/or malformation is seborrheic keratosis; acquired acanthosis nigricans; Becker's nevus; or congenital non-epidermolytic epidermal nevi.
13. The method of any of the preceding paragraphs, wherein the proteasomal inhibitor and/or cationic ionophore is administered topically.
14. The method of any of the preceding paragraphs, wherein the proteasomal inhibitor and/or cationic ionophore is administered intradermally.
15. The method of any of the preceding paragraphs, wherein the proteasomal inhibitor and/or cationic ionophore is administered iontophoretically.
16. The method of any of the preceding paragraphs, wherein the proteasomal inhibitor and/or cationic ionophore is administered systemically.
17. The method of any of the preceding paragraphs, wherein the proteasomal inhibitor and/or cationic ionophore is administered at a dose that does not substantially affect the survival of normal keratinocytes or normal skin explants.

Some embodiments of the technology described herein can be defined according to any of the following numbered paragraphs:

1. A method of treating a benign skin tumor and/or malformation in a subject in need thereof, the method comprising administering a proteasomal inhibitor and Zn2+ or Cu2+ to the subject.
2. The method of paragraph 1, wherein the proteasomal inhibitor is a cationic ionophore.
3. A method of treating a benign skin tumor and/or malformation in a subject in need thereof, the method comprising administering a cationic ionophore to the subject.
4. The method of any of the preceding paragraphs, wherein the cationic ionophore is disulfiram (DSF) or pyrithione.
5. The method of any of the preceding paragraphs, wherein the cationic ionophore is not complexed with Zn2+ or Cu2+.
6. The method of any of the preceding paragraphs, wherein the cationic ionophore administered with Zn2+ or Cu2+.
7. The method of any of the preceding paragraphs, wherein the proteasomal inhibitor and/or cationic ionophore is complexed with Zn2+ or Cu2+.
8. The method of paragraph 6, wherein the cationic ionophore is complexed with Zn2+ or Cu2+ at a ratio of about 1:10 or greater.
9. The method of paragraph 6, wherein the cationic ionophore is complexed with Zn2+ or Cu2+ at a ratio of about 1:1 or greater.
10. The method of paragraph 6, wherein the cationic ionophore is complexed with Zn2+ or Cu2+ at a ratio of about 1:1 to about 10:1.
11. The method of any of the preceding paragraphs, wherein the cationic ionophore is DSF complexed with or administered with Cu2+.
12. The method of any of the preceding paragraphs, wherein the benign skin tumor and/or malformation is seborrheic keratosis; acquired acanthosis nigricans; Becker's nevus; or congenital non-epidermolytic epidermal nevi.
13. The method of any of the preceding paragraphs, wherein the proteasomal inhibitor and/or cationic ionophore is administered topically.
14. The method of any of the preceding paragraphs, wherein the proteasomal inhibitor and/or cationic ionophore is administered intradermally.
15. The method of any of the preceding paragraphs, wherein the proteasomal inhibitor and/or cationic ionophore is administered iontophoretically.
16. The method of any of the preceding paragraphs, wherein the proteasomal inhibitor and/or cationic ionophore is administered systemically.
17. The method of any of the preceding paragraphs, wherein the proteasomal inhibitor and/or cationic ionophore is administered at a dose that does not substantially affect the survival of normal keratinocytes or normal skin explants.
18. A pharmaceutical composition comprising a proteasomal inhibitor and Zn2+ or Cu2+.
19. A composition for the treatment of a benign skin tumor and/or malformation, the composition comprising a proteasomal inhibitor and optionally, Zn2+ or Cu2+.
20. The composition of any of paragraphs 18-19, wherein the proteasomal inhibitor is a cationic ionophore.
21. The composition of any of paragraphs 18-20, wherein the cationic ionophore is disulfiram (DSF) or pyrithione.
22. The composition of any of paragraphs 18-21, wherein the proteasomal inhibitor and/or cationic ionophore is complexed with Zn2+ or Cu2+.
23. The composition of paragraph 22, wherein the proteasomal inhibitor and/or the cationic ionophore is complexed with Zn2+ or Cu2+ at a ratio of about 1:10 or greater.
24. The composition of paragraph 22, wherein the proteasomal inhibitor and/or the cationic ionophore is complexed with Zn2+ or Cu2+ at a ratio of about 1:1 or greater.
25. The composition of paragraph 22, wherein the proteasomal inhibitor and/or the cationic ionophore is complexed with Zn2+ or Cu2+ at a ratio of about 1:1 to about 10:1.
26. The composition of any of paragraphs 18-25, wherein the cationic ionophore is DSF complexed with or administered with Cu2+.
27. The composition of any of paragraphs 19-26, wherein the benign skin tumor and/or malformation is seborrheic keratosis; acquired acanthosis nigricans; Becker's nevus; or congenital non-epidermolytic epidermal nevi.
28. The composition of any of paragraphs 18-27, formulated for topical, intradermal, iontophoretic, or systemic administration.

### EXAMPLES

### Example 1

Seborrheic keratoses (SKs) are common, benign age-related skin tumors. They can be mimic more serious skin conditions such as squamous cell carcinoma and melanoma. Many patients develop multiple lesions and they are a serious cosmetic concern. High activity of the survival kinase Akt underlies most, if not all, of these lesions. Inhibition of Akt leads to dramatic decrease in SK viability.

One important effect of Akt inhibition is an increase in p53 levels. Akt regulates the activity of the p53-suppressor MDM2, which ubiquitinylates p53 and targets it for degradation in the proteosome. The MDM2 inhibitor nutlin-3a was found to also impair SK viability. In the process of studying this process, the activity of other proteasome inhibitors was examined.

As described herein, disulfiram (DSF) with or without copper (Cu) efficiently kills SK cells in vitro. DSF inhibits the chymotrypsin-like activity of the proteasome. In addition, DSF is a cationic ionophore and increases intracellular levels of Cu. This latter activity leads to increase in reactive oxygen species (ROS) concentration, which is not tolerated by tumor cells.

Described herein is a therapeutic window that limits adverse effects on normal keratinocytes. DSF chelates Cu in solution in a 1:1 ratio. This ratio is also the ideal ratio for killing SK cells and sparing normal keratinocytes. Similar activity is demonstrated herein for pyrithione. These agents are ideal for development in the dermatologic sphere since they are already approved for other indications in humans and are very safe at levels beyond what is needed for treating SKs topically. This is in contradistinction to specific Akt inhibitors which are expensive, not FDA-approved and systemically toxic in pre-clinical animal testing.

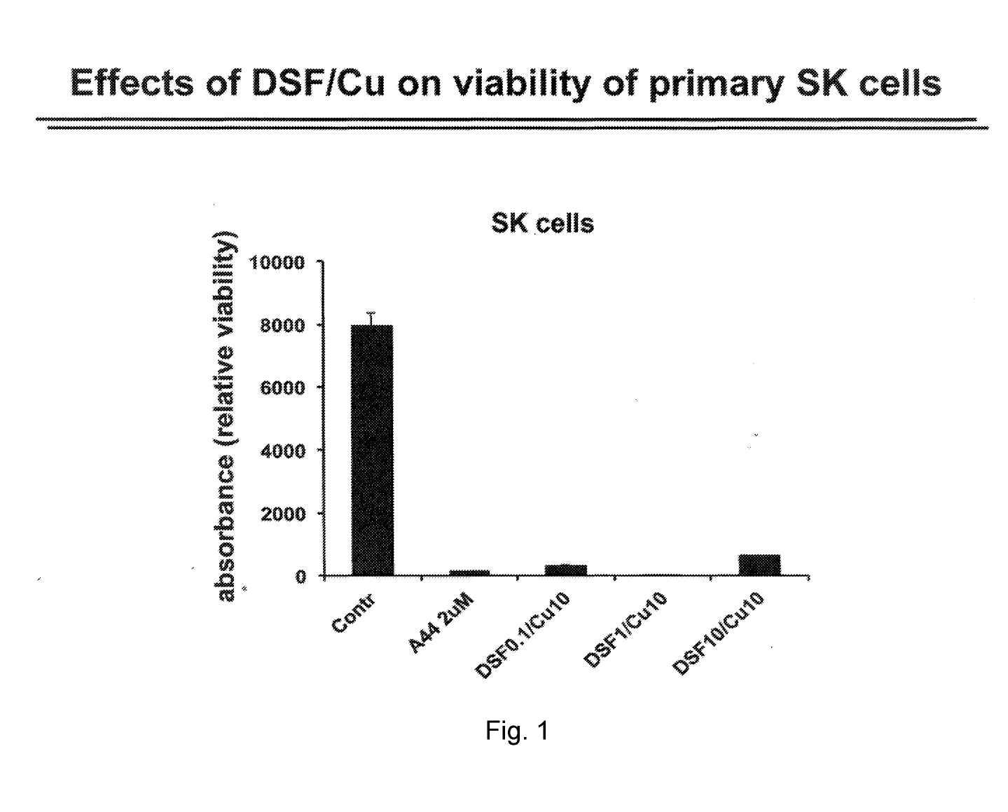

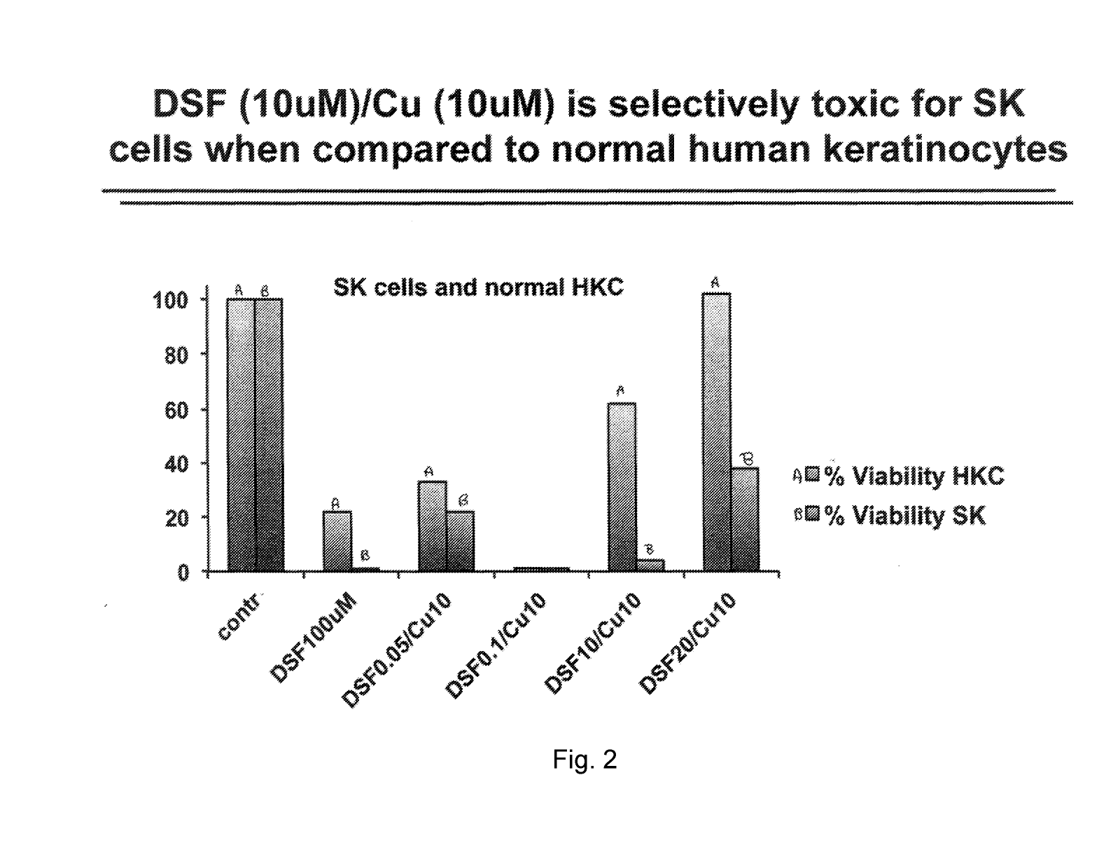

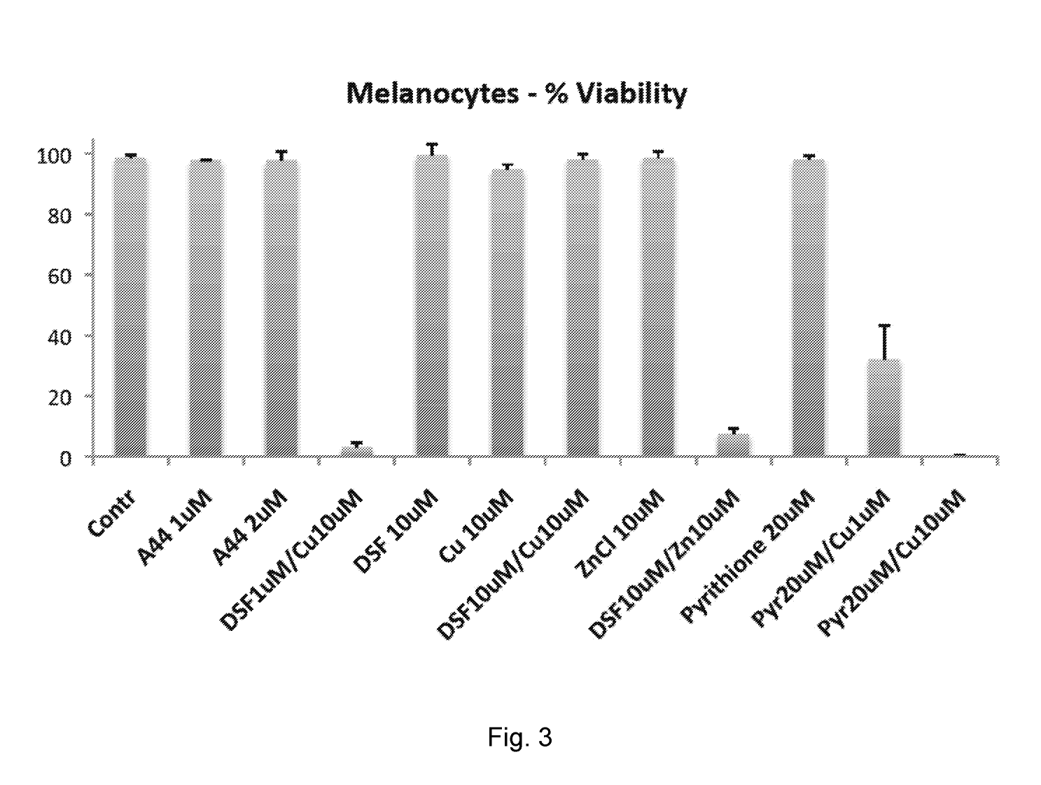

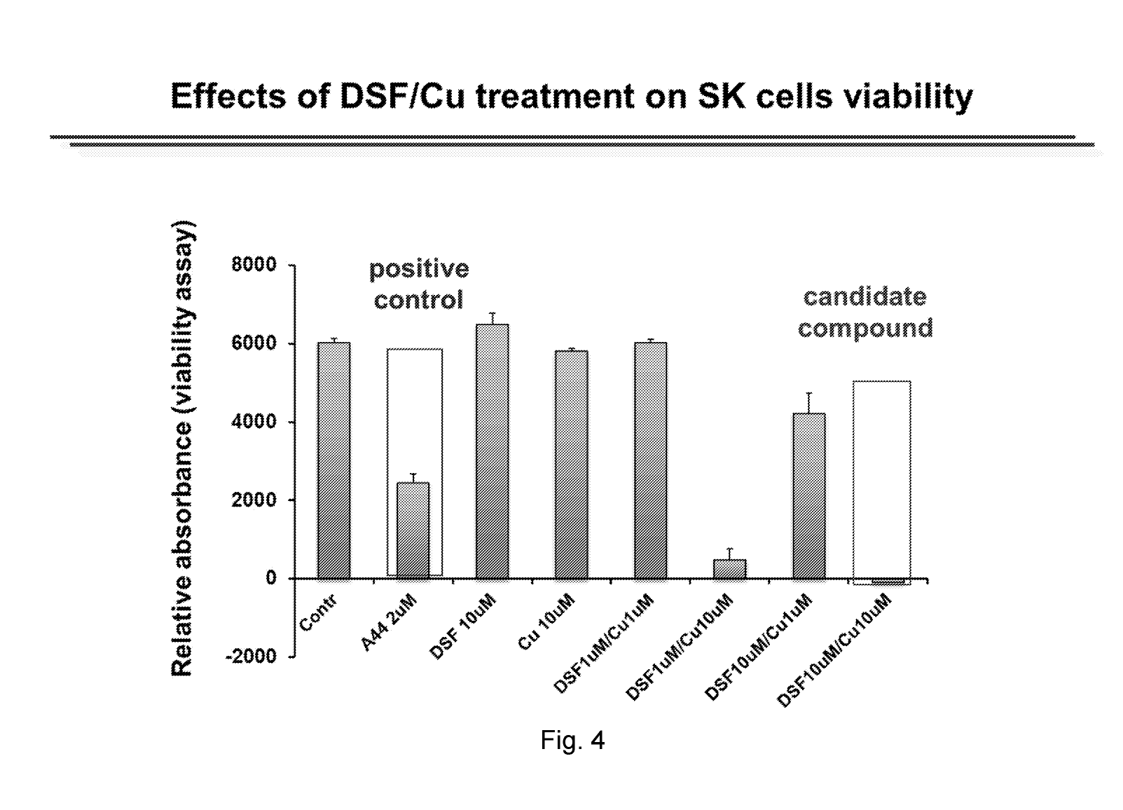

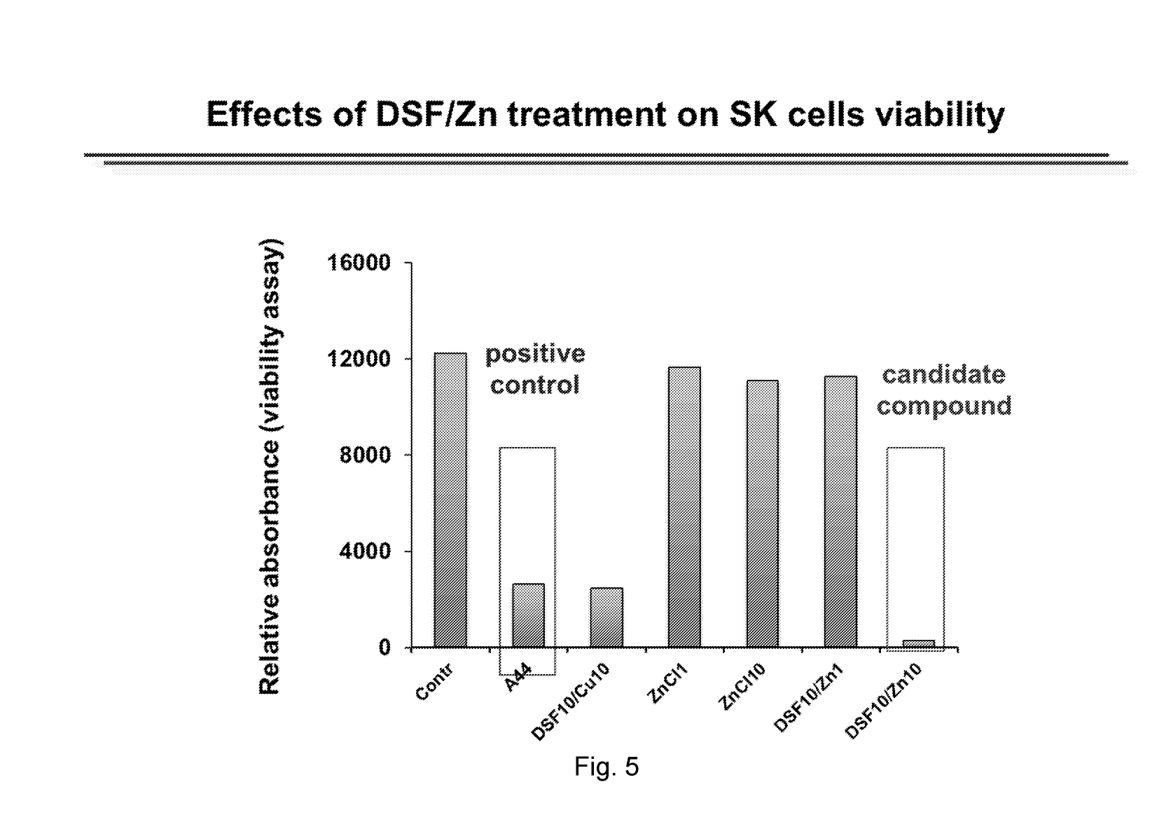

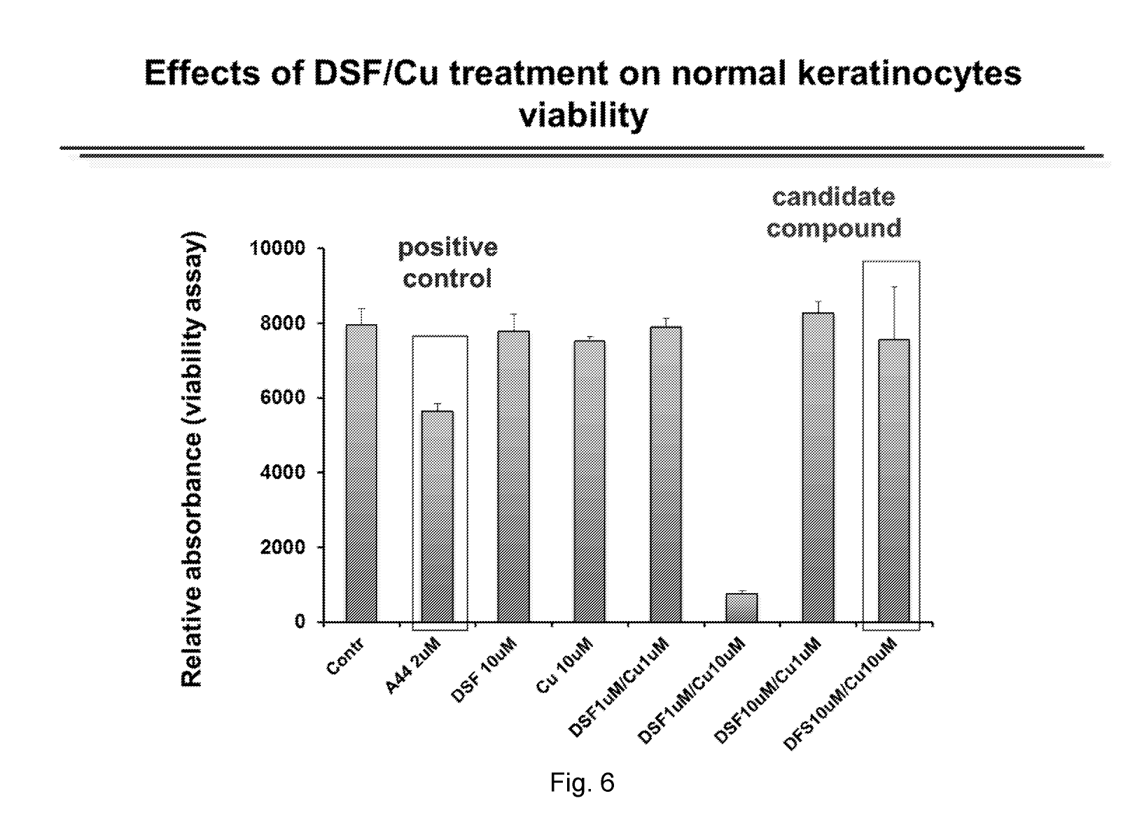

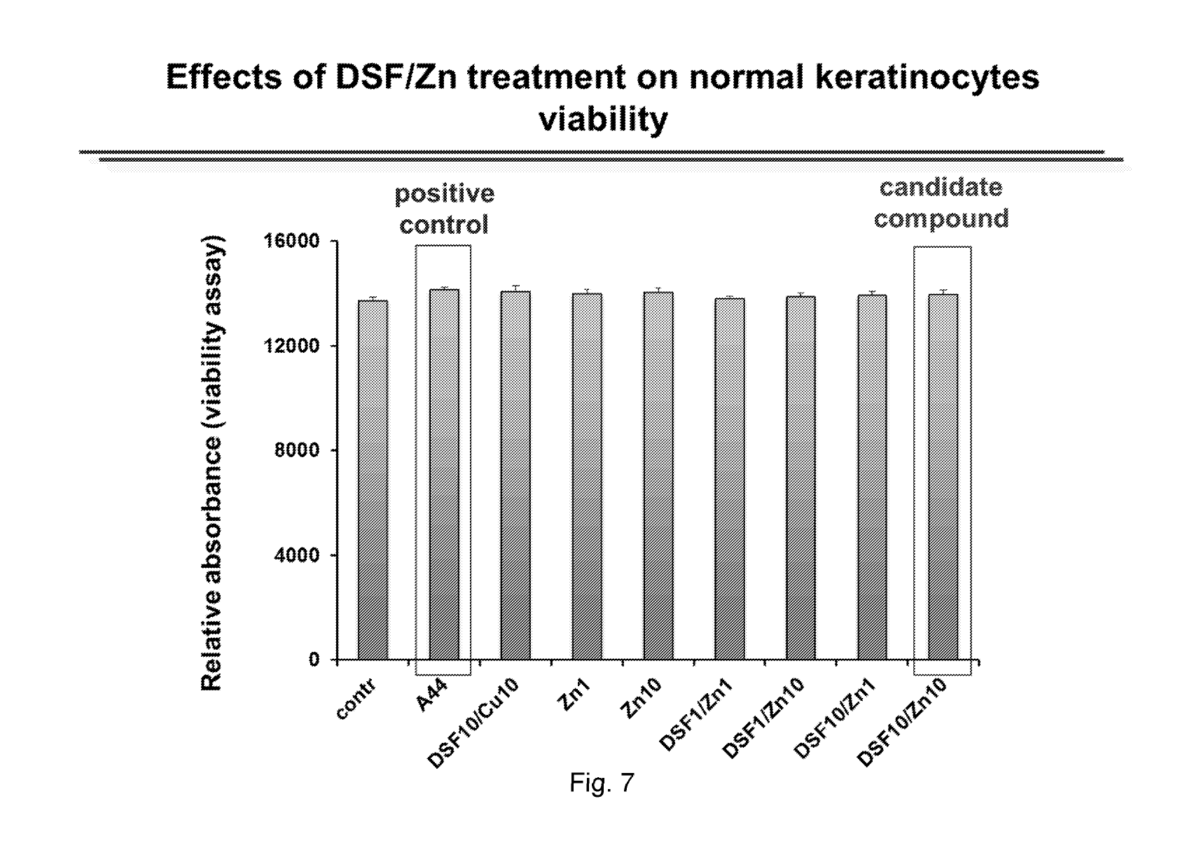

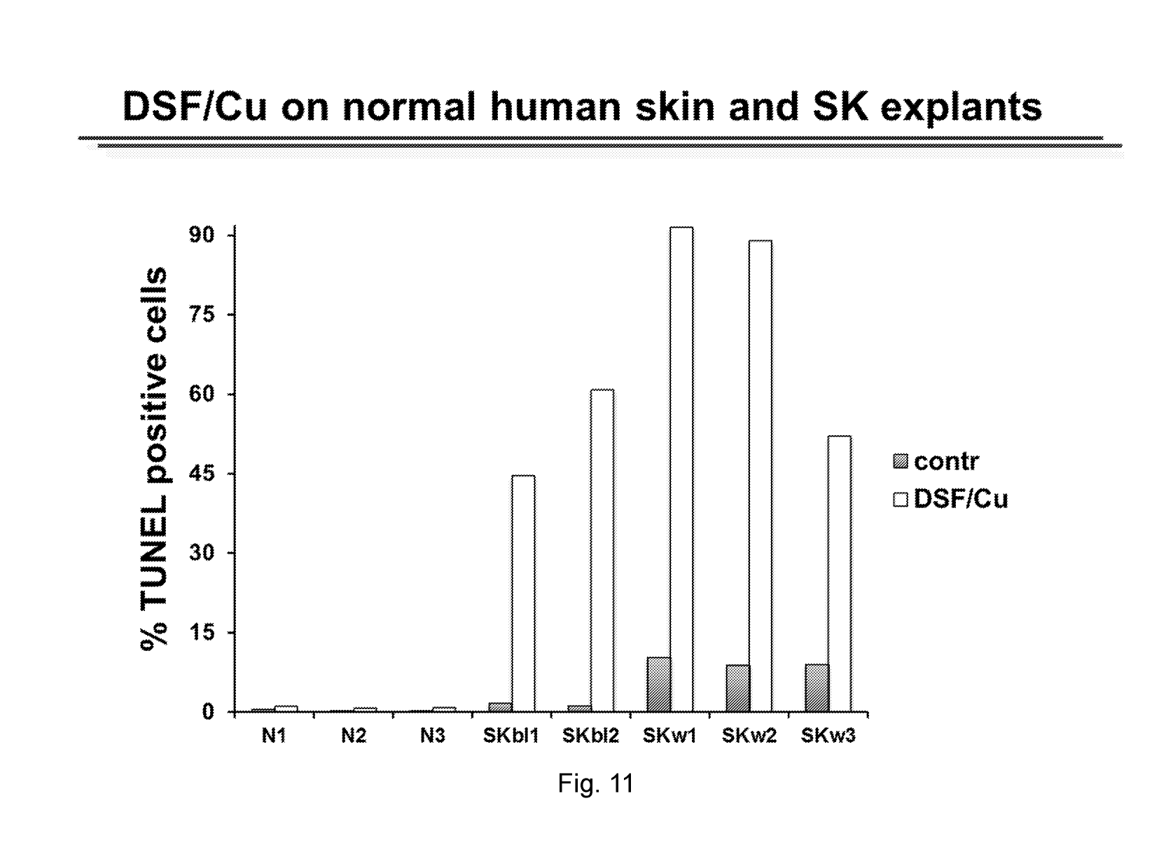

DSF in combination with Cu2+ or Zn2+ was demonstrated to have an inhibitory effect on SK cell viability (FIGS. 4 and 5; FIG. 1) while being nontoxic to normal keratinocytes (FIGS. 6 and 7; FIG. 2). Similar effects were observed for normal skin explants and SK explants (FIG. 11). DSF in combination with Cu2+ at a 1:1 ratio (10 uM:10 uM) was toxic to SK cells but nontoxic for both keratinocytes and melanocytes (FIG. 3). DSF in combination with Zn2+ at a 1:1 ratio (10 uM:10 uM) was toxic to SK cells but nontoxic for keratinocytes (FIG. 3).

Toxicity of DSF in combination with Cu2+ at a 1:1 ration (10 uM:10 uM) was also observed for multiple individual SK explants obtained from both black and white skin as assessed by TUNEL staining and microscopy analysis (data not shown). The same dosage combination was non-toxic to normal skin explants.

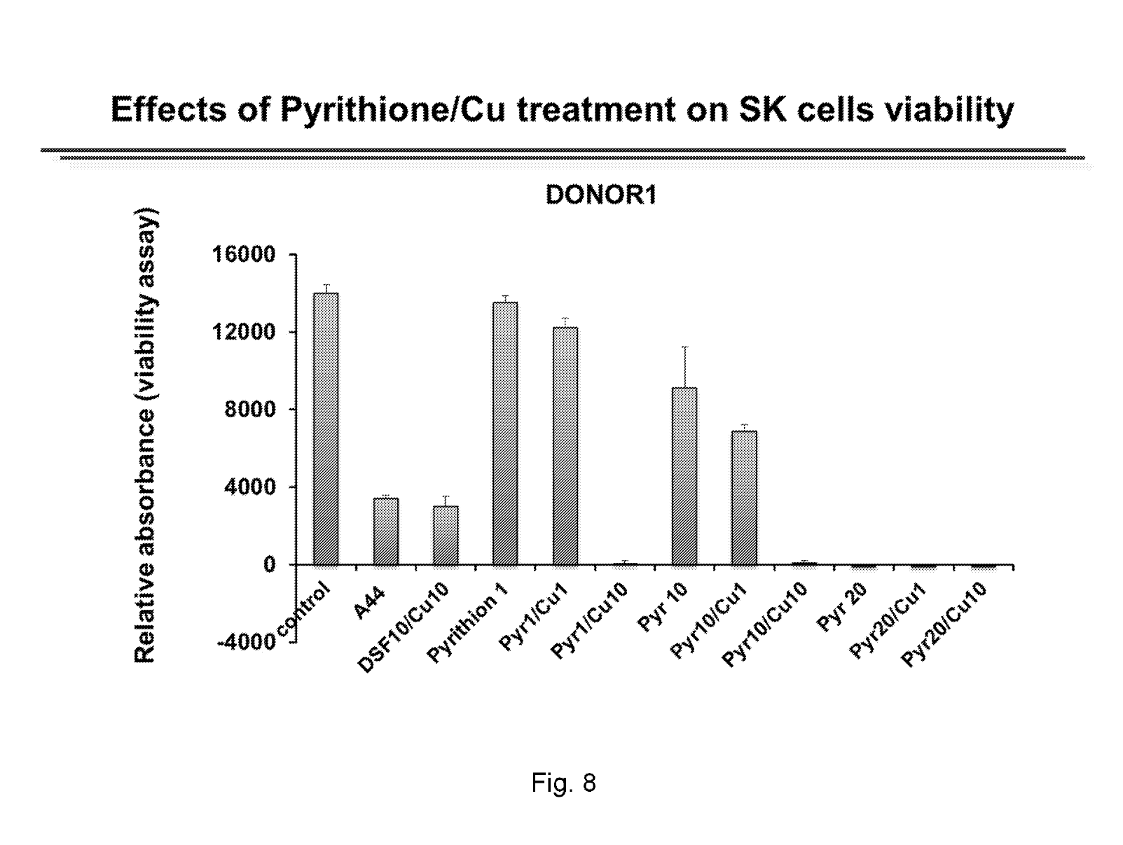

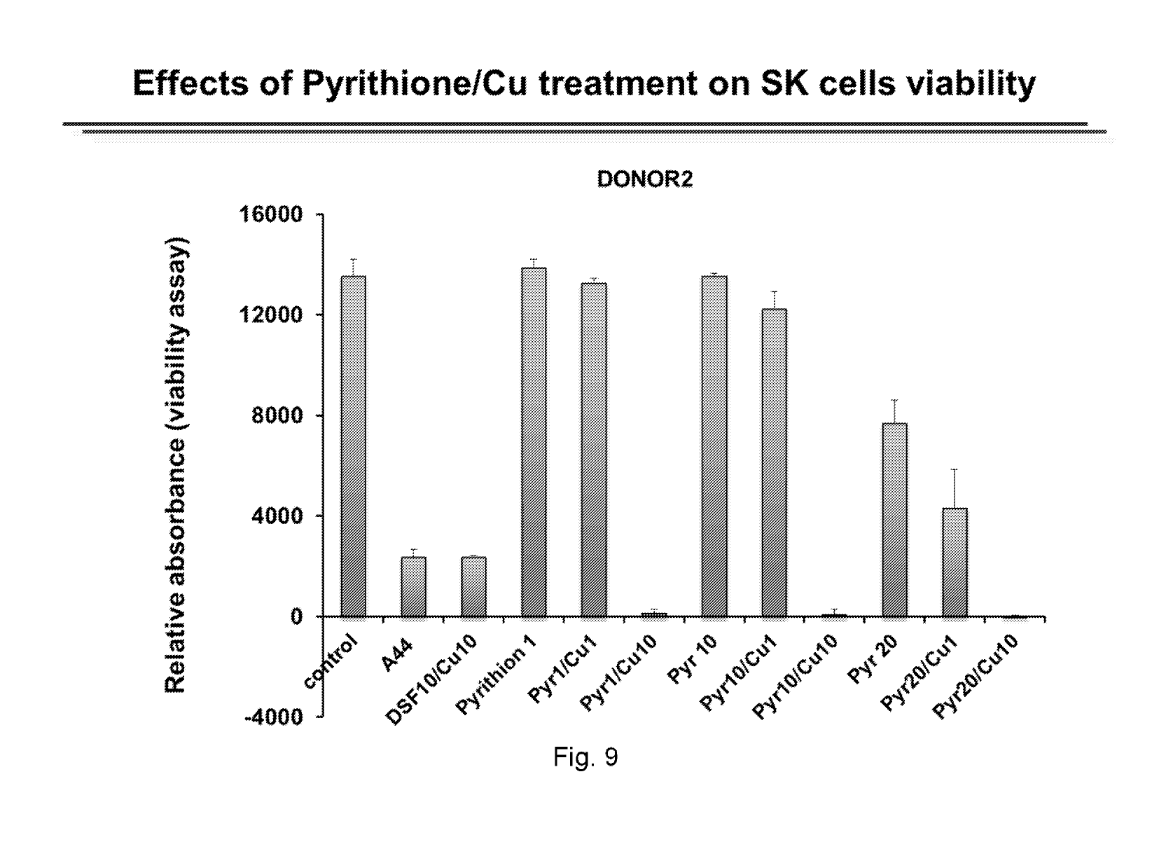

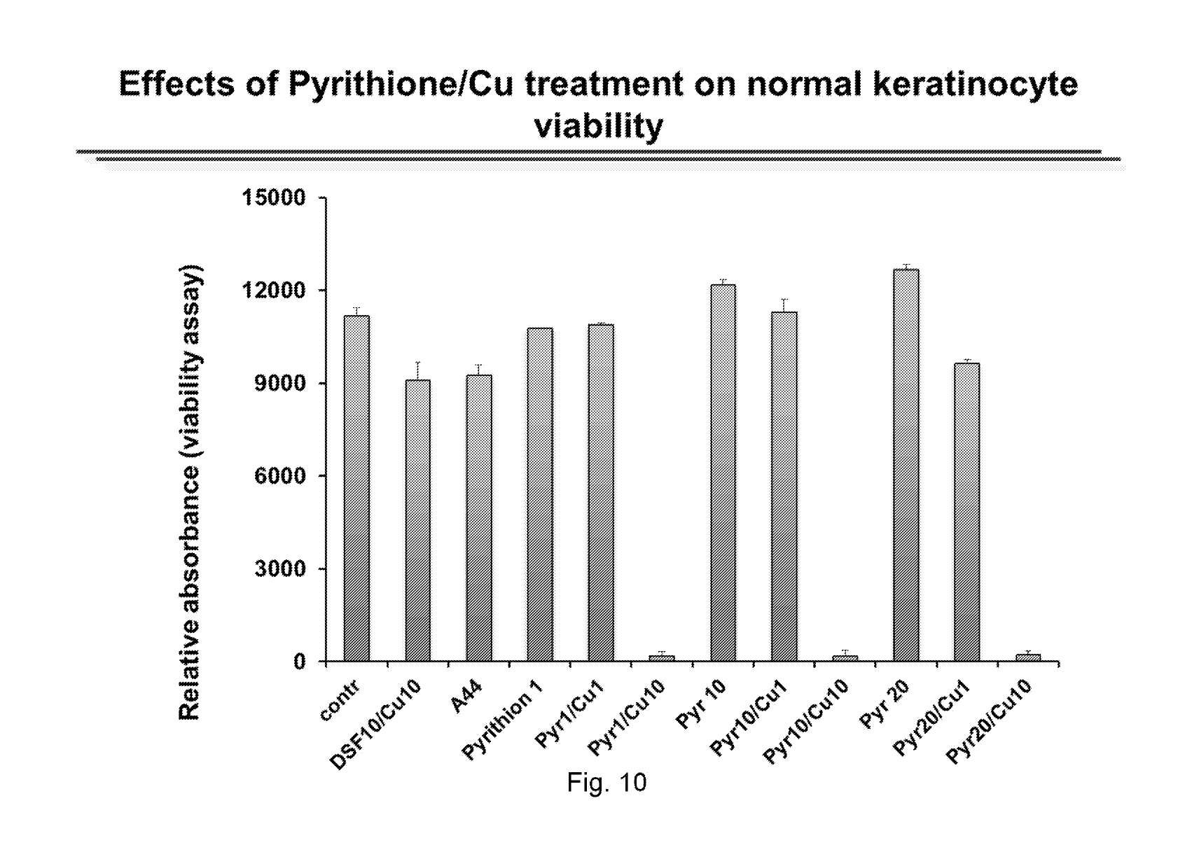

Pyrithione and Cu2+ were demonstrated to have an inhibitory effect on SK cells (FIGS. 8 and 9) while being nontoxic to normal keratinocytes (FIG. 10).

1. A method of treating a benign skin tumor and/or malformation in a subject in need thereof, the method comprising administering a proteasomal inhibitor and Zn2+ or Cu2+ to the subject.

2. The method of claim 1, wherein the proteasomal inhibitor is a cationic ionophore.

3. A method of treating a benign skin tumor and/or malformation in a subject in need thereof, the method comprising administering a cationic ionophore to the subject.

4. The method of claim 2, wherein the cationic ionophore is disulfiram (DSF) or pyrithione.

5. The method of claim 2, wherein the cationic ionophore is not complexed with Zn2+ or Cu2+.

6. The method of claim 2, wherein the cationic ionophore administered with Zn2+ or Cu2+.

7. The method of claim 1, wherein the proteasomal inhibitor and/or cationic ionophore is complexed with Zn2+ or Cu2+.

8. The method of claim 6, wherein the cationic ionophore is complexed with Zn2+ or Cu2+ at a ratio of about 1:10 or greater.

9. The method of claim 6, wherein the cationic ionophore is complexed with Zn2+ or Cu2+ at a ratio of about 1:1 or greater.

10. The method of claim 6, wherein the cationic ionophore is complexed with Zn2+ or Cu2+ at a ratio of about 1:1 to about 10:1.

11. The method of claim 2, wherein the cationic ionophore is DSF complexed with or administered with Cu2+.

12. The method of claim 1, wherein the benign skin tumor and/or malformation is seborrheic keratosis; acquired acanthosis nigricans; Becker's nevus; or congenital non-epidermolytic epidermal nevi.

13. The method of claim 1, wherein the proteasomal inhibitor and/or cationic ionophore is administered topically.

14. The method of claim 1, wherein the proteasomal inhibitor and/or cationic ionophore is administered intradermally.

15. The method of claim 1, wherein the proteasomal inhibitor and/or cationic ionophore is administered iontophoretically.

16. The method of claim 1, wherein the proteasomal inhibitor and/or cationic ionophore is administered systemically.

17. The method of claim 1, wherein the proteasomal inhibitor and/or cationic ionophore is administered at a dose that does not substantially affect the survival of normal keratinocytes or normal skin explants.

18. A pharmaceutical composition comprising a proteasomal inhibitor and Zn2+ or Cu2+.

19.-28. (canceled)
